(19) United States
(12) Patent Application Publication
(10) Pub. No.: US 2005/0036947 A1
(43) Pub. Date: Feb. 17, 2005

(54) Target-specific activatable polymeric imaging agents

(75) Inventors: Egidijus Edward Uzgiris, Schenectady, NY (US); Mohan Mark Amaratunga, Clifton Park, NY (US)

Correspondence Address: General Electric Company, CRD Patent Docket Rm 4A59, P.O. Box 8, Bldg. K-1, Schenectady, NY, 12301, US

(73) Assignee: General Electric Company

(21) Appl. No.: 10/638,888

(22) Filed: Aug. 12, 2003

(51) Int. Cl. A61K 49/00

(52) U.S. Cl. 424/9.34; 530/400

(57) ABSTRACT

A target-specific image-enhancing agent for medical imaging comprises an extended poly(amino acid), wherein at least 90 percent of the amino acid residues are conjugated to signal-generating moieties attached to signal-controlling moieties via bonds that are cleavable by a physiological substance produced by the target. The image-enhancing agent becomes activated when the bonds is cleaved by the physiological substance. The image-enhancing agent is used in detecting and/or diagnosing a disease that is characterized by an overproduction of the substance.

### BACKGROUND OF THE INVENTION

The present invention relates to polymeric imaging agents. In particular, the present invention relates to polymeric imaging agents that are specific to and activatable at a target. The present invention also relates to target-specific activatable polymeric magnetic resonance imaging agents that include therapeutic moieties.

Early detection of diseases and therapeutic intervention are a foundation for improving the survivability of patients contracting serious diseases. Magnetic resonance imaging (“MRI”) has recently become an effective method for detecting conditions associated with many illnesses. MRI is a method of producing images of biological tissues that are exposed to high magnetic fields and radio frequency. The subject undergoing observation is placed in a strong magnetic field, and the protons of the water molecules in the subject are excited with a pulse of radio frequency (“RF”) radiation to produce a net oscillating magnetization in the subject. Various magnetic field gradients and RF pulses then act on the spins to code spatial information into the recorded signals, which are assembled into structural images of the subject.

The relaxation of the spins of water protons undergoing RF excitation in a magnetic field varies from tissue to tissue, leading to differentiable contrast in magnetic resonance (“MR”) images. MR contrast-enhancing agents have been developed to be administered into the subject before imaging in order to enhance the contrast between different regions of the tissue. Many of these contrast-enhancing agents are of the low molecular-weight types, which tend to be cleared rapidly from the body, requiring the completion of the imaging procedure within a very short time after such agents are administered into the patient. Other agents having higher molecular weights tend to be excluded from narrow passages, reducing their benefit in the imaging procedure in addition, an overwhelming majority of prior-art contrast-enhancing agents are non-specific in the sense that they are distributed throughout the region under observation, leading to a challenging task of recognizing diseased regions.

Therefore, there is a continued need to provide imaging agents, in general, and MRI contrast-enhancing agents, in particular, which are specific to diseased regions (“target-specific contrast-enhancing agents”) and remain longer in the circulation to provide ample time for completion of the imaging procedure. In addition, it is very desirable to provide such imaging agents that deliver to a specific target a large number of signal-enhancing moieties. Moreover, it is also very desirable to provide an imaging method that can diagnose specific diseases.

### SUMMARY OF THE INVENTION

In general, the present invention provides agents that are specific to and activatable by a target to become at least functionally more effective. The term “activatable” is used in this disclosure to mean being capable of being transformed or otherwise changed to a functionally more effective state. Such agents, in the functionally more effective state, provide enhanced signals that are detectable by a variety of imaging techniques, such as MRI or fluorescence spectroscopy. The enhanced signals result in an enhanced image of a region under observation. A target-specific image-enhancing agent of the present invention comprises: a poly(amino acid) backbone, wherein at least 90 percent of amino acid residues of the poly(amino acid) are conjugated to signal-generating moieties; and a plurality of cleavable signal-controlling moieties attached to the signal-generating moieties via bonds that are cleavable substantially at a target by a physiological target substance produced by the target; wherein the target-specific image-enhancing agent is rendered functionally more effective when the signal-controlling moieties are cleaved from the signal-generating moieties.

In one aspect, the present invention provides target-specific MRI contrast-enhancing agents and an MRI method for detecting specific diseases. An MRI contrast-enhancing agent of the present invention is activatable substantially at a location of a disease in the body of a subject. In one embodiment of the present invention, the functionally more effective state is one that provides MR images having an enhanced contrast. The phrase “substantially at a location of a disease” means “at or near a location of a disease” such that an expression of the disease can activate the contrast-enhancing agent.

An MRI contrast-enhancing agent of the present invention comprises an extended poly(amino acid) conjugated to chelating moieties that form coordination complexes with paramagnetic ions. The paramagnetic ions are substantially shielded from the protons of surrounding water by blocking moieties that are attached to the chelating moieties via linkers that are cleavable substantially at a location of a disease. When the blocking moieties are cleaved from the chelating moieties at the linker, the paramagnetic ions are exposed to and act on the protons of the surrounding water to provide enhanced contrast in MR images.

In one aspect of the present invention, a blocking moiety is cleaved from a chelating moiety by a physiological target substance (e.g., a physiological substance produced by a target intended for the contrast-enhancing agent when the imaging technique is MRI).

In another aspect of the present invention, the cleavable linker is a specific substrate for an enzyme that is specific to a disease to be diagnosed.

In another aspect of the present invention, each of the chelating moieties comprises a plurality of carboxylic acid groups.

In still another aspect of the present invention, at least 90 percent of the amino acid residues of the poly(amino acid) are conjugated to the chelating moieties.

In still another aspect of the present invention, the MRI contrast-enhancing agent further comprises at least a therapeutic agent for the disease, said at least a therapeutic agent being attached to the MRI contrast-enhancing agent via a linker that is cleavable at or near a location of the disease.

The present invention also provides a method for detecting or diagnosing a disease using an imaging technique and an image-enhancing agent. The method comprises: administering into a subject at least an image-enhancing agent that is specifically activatable by an expression of the disease; and obtaining at least an image, before and after said step of administering, of a portion of the body of the subject, which portion is suspected to carry the disease. The method further comprises locating an area of the MR image obtained after the step of administering, which area of the image embodies an enhanced signal generated by the presence of the image-enhancing agent in its activated state, indicating the presence of the disease. In one aspect, the image-enhancing agent used in the method is an MRI contrast-enhancing agent and comprises an extended poly(amino acid) conjugated to chelating moieties that form coordination complexes with paramagnetic ions. The paramagnetic ions are substantially shielded from the protons of surrounding water by blocking moieties that are attached to the chelating moieties via linkers that are cleavable substantially at a location of a disease. When the blocking moieties are cleaved from the chelating moieties at the linker, the paramagnetic ions are exposed to and act on the protons of the surrounding water to provide enhanced contrast in MR images.

Other features and advantages of the present invention will be apparent from a perusal of the following detailed description of the invention and the accompanying drawings in which the same numerals refer to like elements.

### BRIEF DESCRIPTION OF THE DRAWINGS

FIG. 1 is an illustration of inter-chain and intra-chain attraction of polypeptides.

FIG. 2 is an illustration of a highly conjugated polypeptide of the present invention.

FIG. 3 is an illustration of an activatable contrast-enhancing agent of the present invention, wherein the exemplary chelating moiety is diethylene triamine pentaacetic acid (“DTPA”), and the backbone chain is poly-L-lysine.

FIG. 4 is an illustration of an activatable contrast-enhancing agent of the present invention, further incorporating a target-specific ligand.

FIG. 5 an illustration of an activatable contrast-enhancing agent of the present invention, further incorporating therapeutic moieties in the side arms.

### DETAILED DESCRIPTION OF THE INVENTION

In general, the present invention provides agents that are specific to and activatable by a target to become at least functionally more effective. In one aspect, the present invention provides target-specific MRI contrast-enhancing agents and an MRI method for detecting specific diseases. An MRI contrast-enhancing agent of the present invention is activatable substantially at a location of a disease in the body of a subject. In another aspect, the MRI contrast-enhancing agent accumulates preferentially (i.e., to a higher concentration) at or near the site of the target.

In the present disclosure, the terms “poly(amino acid)” and “polypeptide” are used interchangeably. The terms “imaging agent” and “image-enhancing agent” are used interchangeably. The term “contrast-enhancing agent” is sometimes abbreviated to “contrast agent.”

A contrast-enhancing agent of the present invention comprises an extended poly(amino acid) conjugated to chelating moieties that form coordination complexes with paramagnetic ions. The paramagnetic ions are substantially shielded from the protons of surrounding water by blocking moieties that are attached, for example, by covalent bonds, to the chelating moieties via linkers that are cleavable substantially at a location of a disease. When the blocking moieties are cleaved from the chelating moieties at the linker, the paramagnetic ions are exposed to and act on the protons of the surrounding water to provide enhanced contrast in MR images.

In one aspect of the present invention, a blocking moiety is cleaved from a chelating moiety by a physiological target substance. For example, in one aspect of the present invention, the cleavable linker is a specific substrate for an enzyme that is produced by a diseased tissue. Thus, when the contrast-enhancing agents migrate to the site of the disease, the enzyme reacts with the linker to liberate the blocking moiety, thereby activating the contrast agent. Consequently, areas of enhanced contrast in the MR image indicate the presence of a diseased tissue.

Conformation of the Polymeric Contrast-Enhancing Agent

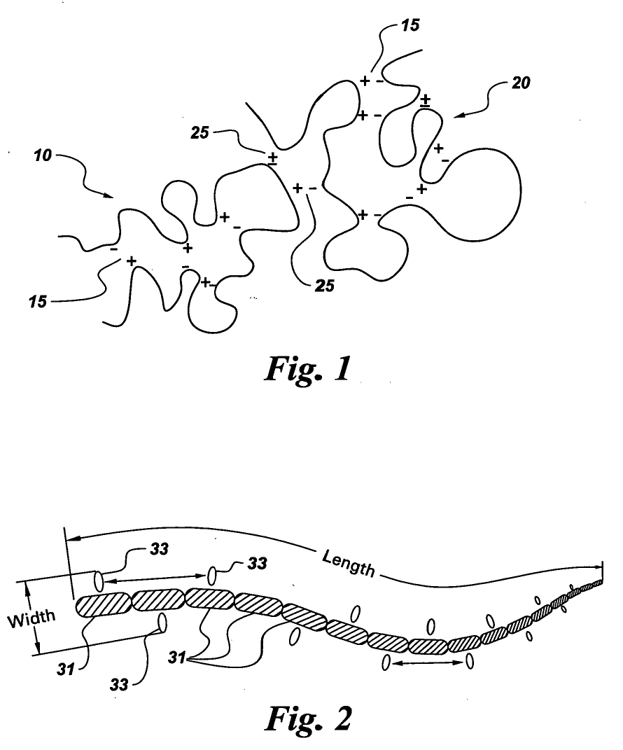

An extended poly(amino acid) contrast-enhancing agent of the present invention has an elongated, worm-like conformation. The conformation of a polymer is a result of interaction of intra-chain charges, which interaction is manifested in the extent of rigidity of the polymer molecule. In general, poly(amino acid) molecules in solution carry opposite charges at the amino and carboxylic acid groups, which interact with each other often to result in a bulky tightly folded or globular conformation. For example, FIG. 1 illustrates two poly(amino acid) chains 10 and 20, each carrying a plurality of positive and negative charges. The segments of the same poly(amino acid) chain 10 or 20 carrying opposite charges attract to each other at 15, resulting in highly folded chains. In addition, opposites charges carried on adjacent chains 10 and 20 also attract to each other at 25, resulting in the formation of large globules, each of which comprises a plurality of chains. On the other hand, a poly(amino acid) chain of the present invention is conjugated, to a large extent, with chelating moieties having net negative charges that inhibit the attraction between segments of the chain so as to result in an elongated conformation. The degree of conjugation of a poly(amino acid) chain of the present invention is at least about 90 percent, preferably at least 95 percent. By “conjugation” or “conjugated,” it is meant in this disclosure that an amino acid residue of the poly(amino acid) chain is attached covalently with at least a portion of another organic molecule, which is the chelator for a cation. Thus, the process of conjugation also includes a process of substitution of at least one atom of an amino acid residue with a portion of the chelator. (The term “residue”, as used in this disclosure, means the remaining portion of a monomeric unit that is linked with portions of other monomeric units to form the polymer.) FIG. 2 illustrates a poly(amino acid) chain comprising amino acid residues 31 linked together through peptide bonds. Each of a very large fraction (greater than 90 percent) of the amino acid residues 31 is conjugated with chelator 33 through a covalent bond. Chelators 33 inhibits, by steric hindrance and charge repulsion, the tendency of the poly(amino acid) to become folded upon itself, resulting a stretched out conformation. Therefore, a contrast-enhancing agent of the present invention can easily enter small pores or spaces, such as a porous space between endothelial cells in an atherosclerotic region of a blood vessel, but at the same time is not easily cleared from the body of the subject. Persistence length is a measure that can quantify the “straightness” of a polymeric chain and is a useful parameter characterizing a contrast-enhancing agent of the present invention. Thus, persistence length can also be viewed as a measure of the degree to which a chain is curved or bent along its contour, or a measure of the stiffness of the chain. Persistence length is the average projection of the end-to-end distance vector (the vector connecting the two ends of the polymer molecule) on the direction of a selected bond vector. The persistence length can be calculated using the radius of gyration of the polymer molecule, which radius of gyration can be determined by a light scattering experiment. See; e.g., Charles R. Cantor and Paul R. Schimmel, “Biophysical Chemistry, Part III: The Behavior of Biological Macromolecules,” pp. 979-1018, W.H. Freeman and Company, New York, N.Y. (1980); Charles R. Cantor and Paul R. Schimmel, “Biophysical Chemistry, Part II: Techniques for the Study of Biological Structure and Function,” pp. 838-846, W.H. Freeman and Company, New York, N.Y. (1980); and Paul J. Flory, “Statistical Mechanics of Chain Molecules,” pp. 36-38, Oxford University Press, New York, N.Y., 1989. The cited sections of these references are incorporated herein by reference. A contrast-enhancing agent of the present invention has a worm-like shape being essentially a stretched-out, extended chain with little folding. A folded poly(amino acid) with little or no conjugation, has a low persistence length of about 10 angstroms, and is not suitable for use in the present invention. On the other hand, a contrast-enhancing agent of the present invention has a persistence length in the range from about 100 to about 600 angstroms. The back-bone chain of a contrast-enhancing agent of the present invention typically has from about 100 to about 650 monomeric amino acid residues.

The conformation of poly(amino acid) chains is also discussed in U.S. Pat. No. 5,762,909; which is incorporated in its entirety in the present disclosure by reference.

In one approach to produce an effective contrast-enhancing agent complex having a proper persistence length, one eliminates or reduces intra-chain charge interactions as well as restricts rotation about a bond at each peptide link. This may be accomplished by making substitution of the chain with a molecule that provides a steric hindrance, extending as side arms from the main chain.

For example, if the polypeptide backbone chain is poly-L-lysine (“PLL”), which has a positive charge at each lysine, one attaches a sufficient amount of substitutions that would impair peptide bond rotation.

One such method is to attach molecules such as diethylene triamine pentaacetic acid (“DTPA”) at most of the lysine residues. Due to the physical size and the steric hindrance effects of DTPA, there is a physical restraint on peptide bond rotation, which restraint extends the polypeptide into a worm-like configuration. Each of these DTPA molecules is attached at an amine group of a lysine amino acid. The degree of substitution is important in defining the conformation of the overall polypeptide. It was found that substituted PLL has high MR imaging efficacy when it is at least 90 percent substituted with DTPA.

In the case that the polypeptide has both positively and negatively charged sections along its length, such as a polypeptide composed of positively charged amino acids having a low degree of substitution with a negatively charged entity, there is a large degree of folding. However, by further substitution, the charge interactions are reduced, thereby reducing the degree of folding.

T₁ Relaxation Time

When the carrier molecule is in an elongated conformation, the chelator entity, which provides the MR activity, is free to rotate about its attachment point to the main chain, allowing a long T₁ relaxation time of the surrounding water protons, which are the source of the MR signal.

When the carrier molecule is in a globular or highly folded conformation, the paramagnetic ions on the chelator entities tumble at a slower rate (along with the entire molecule). Hence, their effect on water protons is to increase their relaxation rate; and, therefore, a shorter T₁ relaxation time results.

It was, therefore, found that a high relaxivity (relaxitivity is the inverse of relaxation time) or short relaxation time is associated with a molecule which folds upon itself into a globular conformation, such as albumin, at about 15 sec⁻¹ milliMolar⁻¹ (sec^{-‘}mM⁻¹). A low relaxivity or long relaxation time is associated with an elongated molecule such as a highly substituted Gd-DTPA-PLL, in which the Gd can rotate rapidly, having a relaxivity of about 8 sec⁻¹ mM⁻¹.

When the relaxivity of a peptide contrast agent was high, the uptake coefficient of such an agent was invariably low, evidently due to the absence of a reptation movement of the contrast agent molecule, resulting in the exclusion of the contrast agent from narrow passages. Thus, it is important to establish that the peptides being compared for optimum length are all of the same conformation. Relaxivity values of the Gd-PLL for various lengths were tested to be between 7.5 and 9.5 for average chain length of 92, 219, 455, 633, and 1163 residues, in a 2 Tesla magnet (2T) at 80 MHz and 23° C. This suggests that a reasonably uniform conformational state was achieved for the peptides being compared.

Charges Carried on the Contrast-Enhancing Agent

Since many in-vivo chemical entities have a negative charge, molecules introduced into the subject must have a net negative charge to reduce agglutination and to allow for stable long circulation in the blood plasma. On the contrary, positively charged molecules tend to stick to cell surfaces, which are generally negatively charged. A high net negative charge is also desirable since it also causes the contrast agent complex molecules to retain their elongated, worm-like conformation.

Preparation of Poly(Amino Acid) Contrast-Enhancing Agents

A wide variety of poly(amino acid) polymers can be used as the backbone chains for synthesis of contrast-enhancing agents of the present invention. The poly(amino acid) can be a homopolymer or a copolymer of at least two types of amino acids. In addition, a wide variety of chelating moieties can be attached to the amino acid residues of the poly(amino acid) backbone chain. The following examples disclose DTPA chelating moiety. However, it should be understood that other polycarboxylic acids that comprise at least a construct of polycarboxylic acid and amine groups can also be used. Such other polycarboxylic acids are disclosed below.

### EXAMPLE 1

### Preparation of Gd-DTPA-PLL Contrast Agent

Under an inert atmosphere, the penta anion of DTPA was prepared by reaction of DTPA (2.97 g, 7.56 mmol) with triethylamine (5.37 ml, 3.9 g, 38.56 mmol) in 35 ml acetonitrile for 50 minutes at 55° C. Isobutylchloroformate (1.10 ml, 1.16 g, 8.47 mmol) was added dropwise to the DTPA penta anion, cooled in an well-equilibrated −45° C. bath, maintained by a Cryotrol temperature controller (Thermo NESLAB, Portsmouth, N.H.). After stirring at this temperature for 1 hour, the resulting thick slurry of the diethylenetriamine tetraaceticacid-isobutyl dianhydride was added dropwise, under ambient atmospheric conditions, to 15 ml of an aqueous 0.1 M NaHCO₃ buffered pH 9 solution of PLL (degree of polymerization (DP)=402, MW=84,000 gmol⁻¹, M_{w}/Mₙ=1.10, 0.25 g, 1.2 mmol lysine residue) at 0° C. (M_{w} is the weight-average molecular weight, and Mₙ is the number-average molecular weight of the polymer.)

After 16 hours of stirring at ambient temperature most (if not all) of the acetonitrile was removed under high vacuum (˜10 microns Hg) over a period of 20 to 25 minutes. A warm water bath was used to maintain uniform temperature, prevent sample bumping, and inhibit vacuum cooling. The resulting solution was centrifuged twice at 5000 rpm and 5° C. to deposit a thick semi-translucent sediment. The supernatant containing the product was purified by dialysis and sometimes further purified by ultrafiltration. The resulting DTPA-polylysine was labeled using hydrated gadolinium citrate at lower pH, such as PH less than 7, preferably less than 6, and more preferably less than 5. Other gadolinium salts, such as gadolinium chloride or gadolinium acetate are also suitable. The efficacy of conjugation was determined by a colorimetric test for the identification of underivatized polylysine amine. Polymer purity was determined by HPLC. Typical values for conjugation ranged from 92-98%. Typical polymer yields raged from 40-60%.

All glassware used in the preparation of the dianhydride was dried by heating under a nitrogen atmosphere. Acetonitrile was distilled from calcium hydride and stored over 4-angstrom molecular sieves. High purity triethylamine and isobutylchloroformate were employed and were stored under inert atmosphere. The dianhydride was prepared in a Morton flask using a mechanical overhead stirrer for achieving high mixing efficiency. Finally, the synthesis up to the DTPA polylysine conjugate was carried out uninterrupted. If necessary, the final polymer can be indefinitely stored at 4° C.

Gd-DTPA-Polylysine Purification

Dialysis:

The polymer solution was loaded into 5 mL regenerated cellulose disposable dialyzers with a molecular weight cut off of 8000 (Sigma-Aldrich catalog number Z36,849-0). The polymer solutions were dialyzed using a Spectra/Por EZ-1 Multidialyzer, against approximately 2 liters of 10 mM NaHCO3 for 24 hours, with constant motion. The buffer was changed after 4-6 hours. The samples were dialyzed for 24 hours. Initial and final dialyzed volumes were noted. The initial and final dialyzed polymer solutions were analyzed by HPLC, without filtering.

Ultrafiltration:

The following devices were used for these experiments: Amicon Centriplus YM-3 centrifugal filter devices, containing a regenerated cellulose membrane with a molecular weight cut off of 3000 (catalog number 4420). The membranes were pre-washed with 50 mM phosphate buffer, pH 7 before use to remove polyethylene glycol. The washing procedure was as follows. Add 14 mL of phosphate buffer to the top of the device. Spin for one hour at 3500 rpm in a Sorvall RC-5B Refrigerated Superspeed Centrifuge refrigerated centrifuge at 10° C. (Dupont Corp., Wilmington Del.). Phosphate buffer from top and bottom of the device was replenished after the washing step. Fresh buffer was added and centrifuged as before. These steps were repeated for a total of four times.

HPLC details:

A Dionex (Sunnyvale, Calif.) DX500 HPLC system equipped with a model PD-40 uv-visible photodiode array detector was used to monitor the synthetic efforts. This system was controlled with Dionex's Peaknet version 5.21 software. For the purposes of this work a Supelco TOSOH Biosep TSK-gel 7.8 mm×30 cm, 10 μM partical size column was utilized. The eluent was 50 mM phosphate buffer, 200 mM NaCl adjusted to pH 7 running at 0.6 ml/min with a run time of 35 minutes.

The conjugated polymers produced by the methods described herein can have a degree of conjugation of about 90 percent or higher. A degree of conjugation of 95 percent or higher has been achieved. Such consistently high degrees of conjugation have not been achieved by other prior art processes. The preferred highly conjugated Gd-DTPA-PLL conjugates exhibit superior relaxivity in bulk water (6.8-7.8 l mol⁻¹ sec⁻¹), as well as penetration in tumor tissues, which also exhibit a high degree of vascular permeability, relative to comparable polymer of lower degrees of conjugation. Such a highly conjugated DTPA-PLL contrast agent has a cross-sectional diameter of about 25 angstroms.

The chemistry and synthesis procedure of Example 1 can be used to prepare contrast agents that comprise, in their backbone chains, residues of amino acids having a free nitrogen-containing group other than lysine, such as histidine, arginine, asparagine, or glutamine. Thus, the poly(amino acid) backbone chain can be polyhistidine, polyarginine, polyasparagine, polyglutamine, or a copolymer of at least two amino acids selected from the group consisting of lysine, histidine, arginine, asparagine, and glutamine.

Specifically, a contrast-enhancing agent of the present invention can comprise a poly(amino acid) selected from the group consisting of polyhistidine, polyarginine, polyasparagine, polyglutamine, and a copolymer of at least two amino acids selected from the group consisting of lysine, histidine, arginine, asparagine, and glutamine, a large fraction (e.g., greater than about 90 percent, preferably greater than 95 percent) of the amino acid residues being conjugated with chelating moieties which form coordination complexes with paramagnetic ions. The chelating moieties can be DTPA or any of the other chelating moieties disclosed below in the section “Other Chelating Moieties.”

### EXAMPLE 2

### Preparation of DTPA-Conjugated Poly(Glutamic Acid)

A method of preparing a poly(glutamic acid) carrier molecule highly substituted with DTPA, which sterically hinders significant folding of the poly(glutamic acid) backbone chain, resulting in a contrast-enhancing agent having worm-like conformation is described below.

A mixed anhydride of DTPA was prepared according to the method as described in P. F. Sieving, A. D. Watson, and S. M. Rocklage, Bioconjugate Chem. Vol. 1, pp. 65-71, (1990).

A flask was charged with 7 ml. acetonitrile and 2.6 g of DTPA. The solution was warmed to 60° C. under a nitrogen atmosphere. Triethylamine was then added via a syringe. The mixture was stirred until homogeneous. The clear solution was then cooled to −30° C. under a nitrogen atmosphere and then 0.5 ml of isobutyl chloroformate was slowly added to result in the anhydride of DTPA.

The anhydride of DTPA is then reacted overnight with ethylene diamine (in which the diamine is in large excess to the anhydride). Ethylene diamine is a suitable choice, giving in the end a DTPA linkage of the desired length to achieve proper steric hindrance against peptide chain bending. The product is separated from the diamine and from DTPA that is not reacted, by ion exchange chromatography. The product has an amine group on one of the acetic acid arms of the pentaacetic acid structure of the DTPA.

Linking this amine-modified DTPA product to the poly(glutamic acid) is done by a carboxyl coupling method. The carboxy acid groups of the poly(glutamic acid) are activated by a coupling reagent, 1-ethyl-3-(3-dimethylaminopropyl) carbodiimide hydrochloride (“EDC”) (Pierce, Rockford, Ill.). The activated group is then combined with the amine-modified DTPA to produce an amide linkage of the DTPA to the peptide backbone as a sidechain which acts as a steric hindrance straightening the polypeptide backbone. The end product is separated by diafiltration.

The resulting poly(glutamic acid) conjugated with DTPA can be converted to Gd-DTPA-poly(glutamic acid) contrast agent by reacting with a gadolinium salt, such as gadolinium citrate, as is disclosed in Example 1.

The chemistry and synthesis procedure of Example 2 can be used to prepare contrast agents that comprise, in their backbone chains, residues of amino acids having a free carboxylic acid group other than glutamic acid, such as aspartic acid. Thus, the poly(amino acid) backbone chain can be poly(aspartic acid) or a copolymer of glutamic acid and aspartic acid.

Specifically, a contrast-enhancing agent of the present invention can comprise a poly(amino acid) selected from the group consisting poly(glutamic acid), poly(aspartic acid), or a copolymer of glutamic acid and aspartic acid; a large fraction (e.g., greater than about 90 percent, preferably greater than 95 percent) of the amino acid residues being conjugated with chelating moieties which form coordination complexes with paramagnetic ions. The chelating moieties can be DTPA or any of the other chelating moieties disclosed below in the section “Other Chelating Moieties.”

In addition, a copolymer of monomeric amino acid residues, each having a free amino group or a free carboxylic acid group, such as a copolymer of lysine and glutamic acid can be used as the backbone chain to prepare a contrast-enhancing agent of the present invention. In this case, an amine-modifed DTPA, such as that prepared according to the procedure of Example 1, would be used to create the chelating moieties extending from the copolymer backbone chain. Such a copolymer is a copolymer of at least a first amino acid selected from the group consisting of lysine, histidine, arginine, asparagine, and glutamine; and at least a second amino acid selected from the group consisting of glutamic acid and aspartic acid.

In one embodiment of the present invention, the poly(amino acid) is a copolymer of lysine and at least one of glutamic acid and aspartic acid.

In another embodiment, a contrast-enhancing agent of the present invention can comprise a poly(amino acid) selected from the group consisting copolymers of at least a first amino acid selected from the group consisting of lysine, histidine, arginine, asparagine, and glutamine; and at least a second amino acid selected from the group consisting of glutamic acid and aspartic acid; a large fraction (e.g., greater than about 90 percent, preferably greater than 95 percent) of the amino acid residues being conjugated with chelating moieties which form coordination complexes with paramagnetic ions. The chelating moieties can be DTPA or any of the other chelating moieties disclosed below in the section “Other Chelating Moieties.”

Other Chelating Moieties

Even though the procedure is illustrated with DTPA, other chelators may also be employed that are capable of being attached to the specific polypeptide being used and that possess a plurality of carboxylic acid groups, which are capable of forming complexes with paramagnetic ions. Non-limiting examples of such other chelators are 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane dietylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

Paramagnetic Ions

Ions that have a partly filled inner shell are suitable to be used in conjunction with a chelator-substituted poly(amino acid) disclosed herein for MRI contrast enhancement. For example, suitable ions are those of transition metal elements, rare earth metals, and actinide elements. Preferred paramagnetic ions are Gd^{3 +}, Dy³⁺, or a mixture thereof.

Blocking Moieties and Linkers

Blocking moieties suitable to be incorporated into a contrast-enhancing agent of the present invention are those that substantially shield the paramagnetic ions from the surrounding water, or otherwise lessen the effect of the paramagnetic ions on the protons of nearby water molecules. Suitable blocking moieties are bulky groups, such as those comprising one or more rings; e.g., substituted or unsubstituted sugars, five-member heterocyclic groups, six-member heterocyclic groups, five-carbon rings, or six-carbon rings, including aryl and cycloalkyl groups. Suitable blocking moieties can comprise at least two and up to ten of these rings connected together by covalent bonds. Alternatively, the linker also can perform the function of a blocking moiety if it can substantially shield the paramagnetic ions when the contrast-enhancing agent is in the unactivated state. In that case, blocking moieties may not be needed.

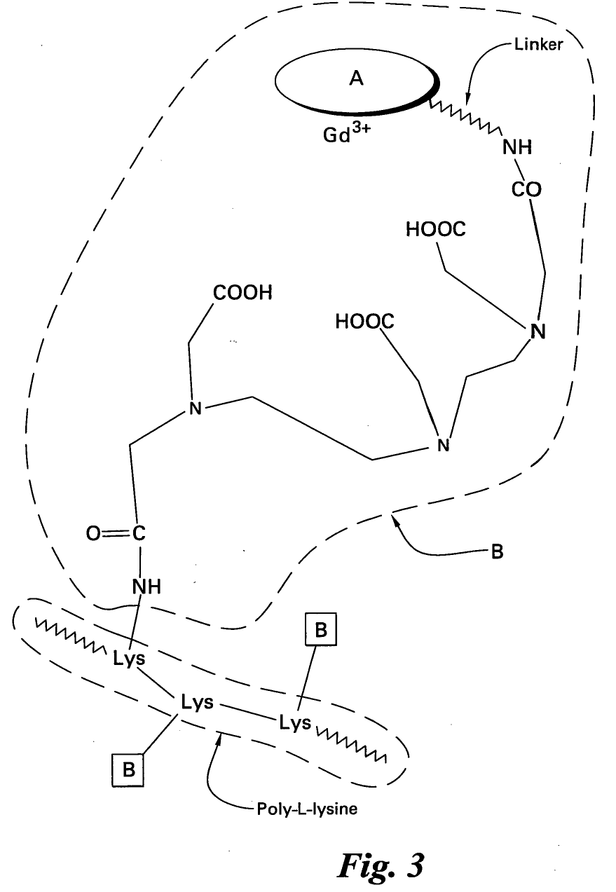

A blocking moiety is attached to a chelating moiety via a cleavable linker. In one embodiment, the cleavable linker can be a substrate, which comprises a peptide, for an enzyme that is produced in excess at a diseased tissue. A higher-than-normal concentration of such an enzyme readily reacts with the linker to liberate the blocking moiety from the contrast-enhancing agent, thus exposing the paramagnetic ions to the protons of the surrounding water to result in an enhanced contrast of the MR image. One end of the linker can be linked to the blocking moiety and the other end of the linker can be linked to the chelating moiety via an amide (—N(R)C(O)— or —C(O)N(R)—), an ester (—OC(O)— or —C(O)O—), an ether (—O—), a ketone (—C(O)—), a thioether (—S—), a sulfinyl (—S(O)—), a sulfonyl (—S(O)₂—), or a direct carbon-carbon bond linkage, wherein R is independently H or a hydrocarbon chain (e.g., alkyl, alkenyl, or alkynyl) having 1 to 14 carbons. For example, when the linker is a poly(amino acid), it can be conveniently and easily attached to the chelating moiety and the blocking moiety via amide linkages of the formula —NH—C(O)—. Any method of making an amide linkage between a peptide and a carboxylic group of a carboxylic acid known in the art can be used. FIG. 3 illustrates an exemplary polymeric contrast-enhancing agent of the present invention; wherein the polymeric backbone is poly-L-lysine; the chelating moiety is DTPA which chelates Gd³⁺ ions, and is attached to a lysine residue via an amide bond; the blocking moiety, denoted by A, is attached to DTPA via the cleavable linker. B denotes the group comprising the chelating moiety with the paramagnetic ion Gd³⁺, the cleavable linker, and the blocking moiety A.

Suitable classes of enzymes that can be targets for contrast-enhancing agents of the present invention include, but are not limited to, hydrolysases such as proteases, carbohydrases, lipases, nucleases, tautomerases, mutases, and transferases.

As will be appreciated by those skilled in the art, the potential list of suitable enzyme targets is quite large. Enzymes associated with the generation or maintenance of atherosclerotic plaques and lesions within the circulatory system, inflammation, wounds, immune response, tumors, may all be detected using the present invention. Enzymes such as lactase, maltase, sucrase or invertase, cellulase, α-amylase, aldolases, glycogen phosphorylase, kinases such as hexokinase, proteases such as serine, cysteine, aspartyl and metalloproteases are also detected, including, but not limited to, trypsin, chymotrypsin, and other therapeutically relevant serine proteases such as tPA and the other proteases of the thrombolytic cascade; cysteine proteases including: the cathepsins, including cathepsin B, D, L, S, H, J, N, and O; calpain; and caspases, such as caspase-1, -3, -5, -6, -8, and other caspases of the apoptotic pathway, and interleukin-converting enzyme (“ICE”). Similarly, bacterial and viral infections may be detected via characteristic bacterial and viral enzymes. As will be appreciated in the art, this list is not meant to be limiting.

Once the target enzyme is identified or chosen, the enzyme substrate serving as the linker in a contrast-enhancing agent of the present invention can be designed using parameters of enzyme substrate specificities.

For example, when the enzyme target substance is a protease, the linker is a peptide or polypeptide, comprising, for example, two to fifteen amino acid residues, which is capable of being cleaved by the target protease.

Similarly, when the enzyme target substance is a carbohydrase, the linker is a carbohydrate group that is capable of being cleaved by the target carbohydrase. For example, when the enzyme target is lactase or β-galactosidase, the linker is lactose or galactose. Similar enzyme/linker pairs include sucrase/sucrose, maltase/maltose, and α-amylase/amylose.

An important class of enzymes that have been associated with many diseases is matrix metalloproteases (“MMPs”). Many pathological conditions are associated with the rapid unregulated breakdown of extracellular matrix tissue by MMPs. Each of the MMP enzymes attacks a specific tissue. For example, MMP-1, MMP-8, and MMP-13 are collagenases. MMP-2 and MMP-9 are gelatinases. MMP-3, MMP-10, and MMP-11 are stromalysins. MMP-14, MMP-15, MMP-16, and MMP-17 are membrane matrix metaloproteases. Some of these pathological conditions include rheumatoid arthritis, osteoarthritis, septic arthritis, corneal, epidermal or gastric ulceration; periodontal disease, proteinuria, coronary thrombosis associated with atherosclerotic plaque rupture and bone disease. The process of tumor metastasis and angiogenesis also appears to be dependent on MMP activity. Since the cycle of tissue damage and response is associated with a worsening of the disease state, limiting MMP-induced tissue damage due to elevated levels of the proteases with specific inhibitors of these proteases is a generally useful therapeutic approach to many of these debilitating diseases. Detecting such proteases, and thus the disease condition, is facilitated with contrast-enhancing agents of the present invention.

In one embodiment, the linker is a peptide substrate for the metalloprotease MMP-2 or MMP-9 (gelatinases). A preferred substrate peptide comprises the sequence Pro-Leu-Gly-Val-Arg. Other suitable sequences include sequences comprising one or more of Pro-Cha-Gly-Cys-His; Pro-Gln-Gly-Ile-Ala-Gly-Gln-D-Arg; Pro-Gln-Gly-Ile-Ala-Gly-Trp; Pro-Leu-Gly-Cys-His-Ala-D-Arg; Pro-Leu-Gly-Met-Trp-Ser-Arg; Pro-Leu-Gly-Leu-Trp-Ala-D-Arg; Pro-Leu-Ala-Leu-Trp-Ala-Arg; Pro-Leu-Ala-Leu-Trp-Ala-Arg; Pro-Leu-Ala-Tyr-Trp-Ala-Arg; Pro-Tyr-Ala-Tyr-Trp-Met-Arg; Pro-Cha-Gly-Nva-His-Ala; Pro-Leu-Ala-Nva; Pro-Leu-Gly-Leu; Pro-Leu-Gly-Ala; Arg-Pro-Leu-Ala-Leu-Trp-Arg-Ser; Pro-Cha-Ala-Abu-Cys-His-Ala; Pro-Cha-Ala-Gly-Cys-His-Ala; Pro-Lys-Pro-Gln-Gln-Phe-Phe-Gly-Leu; Pro-Lys-Pro-Leu-Ala-Leu; Arg-Pro-Lys-Pro-Tyr-Ala-Nva-Trp-Met; Arg-Pro-Lys-Pro-Val-Glu-Nva-Trp-Arg; Arg-Pro-Lys-Pro-Val-Glu-Nva-Trp-Arg; and Arg-Pro-Lys-Pro-Leu-Ala-Nva-Trp. These sequences identify the natural amino acid residues using the customary three-letter abbreviations (see; e.g., C. K. Mathews, K. E. van Holde, and K. G. Ahern, “Biochemistry,” p. 129, Addison Wesley Longman, San Francisco (2000) for the abbreviations of the names of amino acids); and the following abbreviations represent the indicated non-natural amino acids: Abu=L-α-aminobutyryl; Cha=L-cyclohexylalanine; and Nva=L-norvaline.

Caspases, which are a class of cysteine proteases, are associated with inflammation and apoptosis. Linkers that recognize these enzymes are incorporated in contrast-enhancing agents to provide better MR images of tissues with these conditions. For example, suitable linkers for caspase-1 are Phe-Glu-Ala-Asp; Tyr-Val-His-Asp; and Leu-Glu-Ser-Asp. Suitable linkers for caspase-3 are Asp-Glu-Val-Asp; Asp-Gly-Pro-Asp; As-Glu-Leu-Asp; As-Glu-Leu-Asp; and Asp-Glu-Glu-Asp. A suitable linker for caspase-6 is Val-Glu-Ile-Asp.

Another protease that has been known to be overexpressed in many tumors is cathepsin D. A two-to-fifty fold increase in the level of this enzyme has been reported in breast cancers. Cathepsin D also participates in the metastatic cascade by activating other proteolytic enzymes. Early identification of the situs of an elevated level of this enzyme would lead to early and efficient treatment of the tumor. A contrast-enhancing agent of the present invention designed to detect this enzyme comprises a linker that comprises the peptide sequence of Pro-Ile-Cys-Phe-Phe-Arg-Leu.

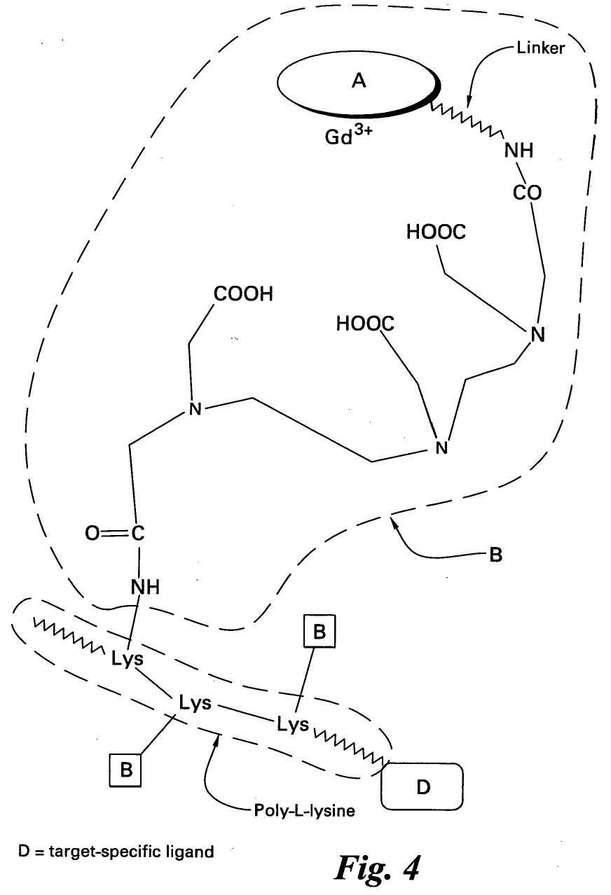

In another embodiment, the specificity of a contrast-enhancing agent of the present invention is enhanced further by including a peptide that binds specifically to receptors on diseased cells. For example, the linear octapeptide having a sequence of EMTOVNOG and the cyclic peptide EMTOVNOGQ have been shown to bind to α-fetoprotein (“AFP”) receptor on MCF-7 human breast cancer cells. Here, the amino acids are represented in a customary manner by single letters, wherein E, M, T, V, N, G, and Q represent glutamic acid, methionine, threonine, valine, asparagines, glycine, and glutamine, respectively; and O represents 4-hydroxyproline. FIG. 4 shows schematically a structure of a contrast-enhancing agent of the present invention incorporating one of these peptides; wherein A denotes a blocking moiety attached to the chelating moiety via a cleavable linker; B denotes the group comprising the chelating moiety with the paramagnetic ion, the cleavable linker, and the blocking moiety; and D denotes a target-specific ligand. It is preferred that the peptide sequence is attached to one end of the polymeric backbone of the contrast-enhancing agent. However, it should be understood that the peptide sequence can be attached to both ends of or internalized in the polymeric backbone. The receptor-specific ligand ensures that the contrast-enhancing agent preferentially concentrates at the diseased tissue. The disease-specific linker on the contrast-enhancing agent further enhances its diagnostic capability, as disclosed above. The receptor-specific ligand is attached to the polymeric backbone, for example, by amide linkages.

The linkers and blocking moieties can be attached to the chelating moieties before or after paramagnetic ions are chelated to the chelating moieties. When the linkers and blocking moieties are attached after the paramagnetic ions are chelated to the chelating moieties, it may be advantageous to contact the contrast-enhancing agent again with a solution containing paramagnetic ions to ensure that the chelating capacity of the chelating moieties is fully used to a substantial extent.

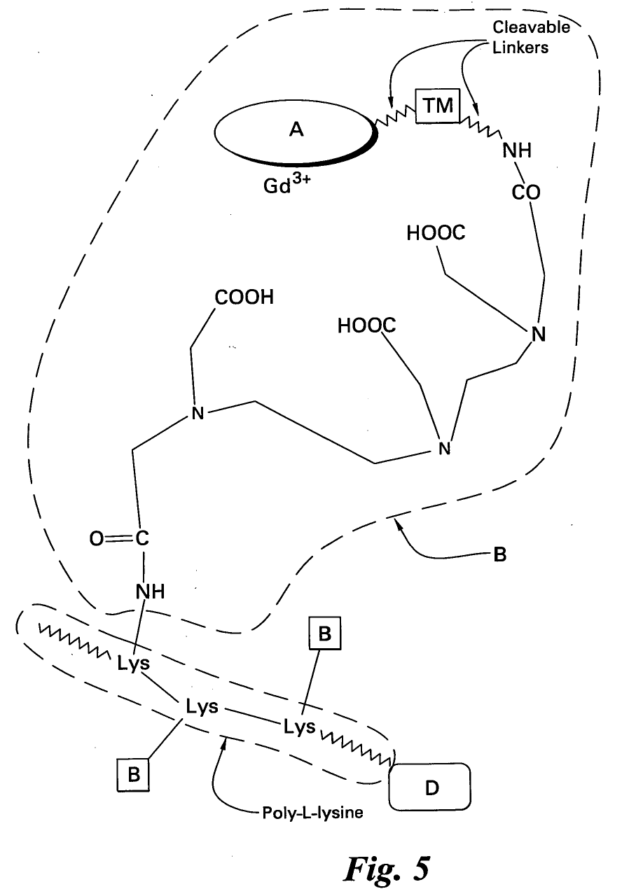

In another embodiment, at least a therapeutic moiety is attached to the polymeric imaging agent via at least a linker that is cleavable by physiological target substances, such as enzymes that are overproduced by a diseased condition. In one example, when an enzyme cleaves the therapeutic moiety from the polymeric imaging agent, the therapeutic moiety binds to, inhibits the action, and limits the damaging effect of the enzyme on the surrounding tissue. For example, FIG. 5 illustrates a polymeric MRI contrast agent of the present invention comprising therapeutic moieties (denoted by “TM”) attached to cleavable linkers on the side arms. Therapeutic moieties also can be attached to the polymeric backbone through cleavable linkers. Thus, a polymeric contrast-enhancing agent of this type can provide the benefit of an enhanced contrast of the MR image of the diseased tissue and, at the same time, and an attenuation of the effect of the root cause of the disease. One type of therapeutic moieties, which are useful as inhibitors of matrix metalloproteinases, are disclosed in U.S. Pat. No. 6,455,570; which is incorporated herein by reference.

In still another embodiment, a target-specific image-enhancing agent is provided which is activatable substantially at the situs of a diseased tissue and is detected by fluorescent spectroscopy. In this embodiment, an optical agent, such as a near-infrared fluorescent dye, is attached via a cleavable linker to the carboxylic acid groups of the polycarboxylic-acid side arms of a polymeric image-enhancing agent. The polycarboxylic-acid side arms can be selected from the group consisting of chelating moieties disclosed herein above. In one example, when the dyes are still attached to the image-enhancing agent, their optical activity is quenched because of their proximity to one another. At the situs of the diseased tissue, the enzyme specific to the linker liberates the dyes from the polymeric chain, thus activates the dyes to provide optical signals. A near-infrared fluorescent agent and a method of optical imaging with this agent were disclosed by Weissleder et al. (see; e.g., R. Weissleder et al., “In Vivo Imaging of Tumors With Protease-Activated Near-Infrared Fluorescent Probes,” Nat. Biotech., Vol. 17, pp. 375-378 (1999); C. Bremmer et al., “Optical Imaging of Matrix Metalloproteinase-2 Activity in Tumors: Feasibility Study in a Mouse Model,” Radiology, Vol. 221, No. 2, pp. 523-529 (2001)).

In still another embodiment, the target-specific image-enhancing agent that comprises an optical agent, as disclosed in the previous paragraph, further comprises at least a therapeutic moiety attached to the polymeric image-enhancing via at least a linker that is cleavable by a physiological target substance, such as an enzyme that is overproduced by a diseased tissue. At the situs of the diseased tissue, the physiological target substance cleaves the therapeutic moiety from the polymeric image-enhancing agent, thereby allowing the therapeutic moiety to treat the disease condition, such as by disrupting a biochemical pathway of the disease expression.

Method of Imaging a Diseased Tissue and Diagnosing the Disease

The present invention also provides a method for detecting and diagnosing a disease condition through imaging a diseased tissue with the assistance of a polymeric imaging agent that is activatable by a substance produced by the disease. In one aspect, the method of the present invention also allows for at least a therapeutic agent, which can be incorporated as a segment on the imaging agent, to be carried to and released at the situs of the disease.

In general, the method for detecting and diagnosing a disease comprises: (a) administering into a subject at least an imaging agent that is specifically activatable by an expression of the disease; and (b) obtaining images, before and after the step of administering, of a portion of the body of the subject, which portion is suspected to carry the disease. When the disease is present in the imaged portion of the body, the image acquired after the imaging agent has been administered into the subject shows an increase in the signal generated by an activation of the agent at the disease locations.

In one aspect, the imaging agent comprises: (1) an extended poly(amino acid), wherein at least 90 percent of the amino acid residues of the poly(amino acid) are conjugated to signal-generating moieties; and (2) a plurality of cleavable signal-controlling moieties attached to the signal-generating moieties via bonds that are cleavable substantially at the target by a physiological target substance produced by the target. The target can be, for example, a diseased tissue. The signal-controlling moieties suppress the signal when they are attached to the imaging agent. When the signal-controlling moieties are cleaved from the imaging agent, the signal is freely expressed.

In one aspect, the imaging agent is an MRI contrast-enhancing agent disclosed herein above. Subtle changes in the images are detected more readily because of an increased contrast brought about by the ability of a contrast agent of the present invention to go through small passages and to collect at the situs of the disease.

In one aspect, the imaging procedure is MRI, the imaging agent is an MRI contrast-enhancing agent, and the increase in the signal results in an enhanced contrast in the MR image compared to an image and a signal acquired before administering the contrast agent. Such an increased contrast and increased MR signal are a result of an increase in MR T₁ relaxation time. For example, an increase in the MR signal of 10 percent or more can signify the presence of the disease in the area under investigation.

In one aspect of the method, the MR contrast-enhancing agent is administered into the subject at a dose in the range from about 0.01 to about 0.05 moles Gd/kg of body weight of the subject. MR images and signals are acquired within 48 hours after the MR contrast agent is first administered into the subject. An MRI system that can be used for practicing a method of the present invention is disclosed in U.S. Pat. No. 6,235,264; which is incorporated herein by reference in its entirety. In one aspect of the present invention, a contrast-enhancing agent is administered intravenously into a subject. A contrast-enhancing agent can also be administered orally under appropriate circumstances.

In one aspect, the method provides a detection of tumor growths associated with a variety of cancers through the use of polymeric contrast-enhancing agents that are activatable by a variety of enzymes, the overproduction of which is associated with the cancers. Non-limiting examples of these enzymes and substrates that recognize them are disclosed herein above.

In another aspect, the present invention provides a method for assessing an effectiveness of a prescribed regimen for treating a disease that is characterized by an overproduction of a disease-specific substance. The method comprises: (a) obtaining at least a base-line image of and acquiring a base-line signal from a portion of a subject, which portion is suspected to carry the disease; (b) administering a first time into a subject a predetermined dose of at least an target-specific image-enhancing agent that comprises an extended poly(amino acid) conjugated to signal-generating moieties that are attached to signal-controlling moieties via bonds that are cleavable by a physiological target substance that is overproduced by a diseased tissue; (c) obtaining pre-treatment images of and acquiring pre-treatment signals coming from the portion of the subject, which portion is suspected to carry the disease, after administering the predetermined dose of the image-enhancing agent into the subject; (d) treating a condition of the disease in the subject with the prescribed regimen; (e) administering a second time into the subject the predetermined dose of said at least an image-enhancing agent; (f) obtaining post-treatment images of and acquiring post-treatment signals coming from the same portion of the subject as in step (c); and (g) comparing post-treatment images and post-treatment signals to pre-treatment images and pre-treatment signals to assess the effectiveness of the prescribed regimen. A decrease in image contrast or signals during the course of the prescribed regimen indicates that the treatment has provided benefit. The method further comprises repeating steps (e), (f), and (g) at predetermined time intervals during the course of treatment of the disease. In one aspect, at least 90 percent of the amino acid residues of the poly(amino acid) are conjugated to the signal-generating moieties. When the signal-controlling moieties are cleaved from the signal-generating moieties, the image-enhancing agent becomes activated.

In another aspect, the imaging procedure is MRI, the imaging agent is an MRI contrast-enhancing agent, wherein the signal-generating moieties comprise chelating moieties that form coordination complexes with paramagnetic ions, and an increase in the signal results in an enhanced contrast in the MR image compared to an image and a signal acquired before administering the contrast agent. Such an increased contrast and increased MR signal are a result of an increase in MR T₁ relaxation time. The signal-controlling moieties, when attached to the signal-generating moieties, comprise blocking moieties that shield the paramagnetic ions from the protons of the surrounding water molecules

During the course of the treatment of the disease, a decreased MR signal (compared to a base-line MR signal obtained before the treatment) of, for example, 10 percent or more can signify that the treatment has conferred some benefit.

In one aspect of the method, the MR contrast-enhancing agent is administered into the subject at a dose in the range from about 0.01 to about 0.5 moles Gd/kg of body weight of the subject. MR images and signals are acquired within 48 hours after the dose of MR contrast agent is administered into the subject.

The prescribed regimen for treating the disease can be, for example, treatment with drugs, radiation, or surgery.

While various embodiments are described herein, it will be appreciated from the specification that various combinations of elements, variations, equivalents, or improvements therein may be made by those skilled in the art, and are still within the scope of the invention as defined in the appended claims.

1. A target-specific image-enhancing agent for medical imaging, the image-enhancing agent comprising:
an extended poly(amino acid), wherein at least 90 percent of amino acid residues of the poly(amino acid) are conjugated to signal-generating moieties; and
a plurality of cleavable signal-controlling moieties attached to the signal-generating moieties via bonds that are cleavable substantially at a target tissue by a physiological target substance produced by the target tissue;
wherein the target-specific imaging-enhancing agent is rendered functionally more effective when the signal-controlling moieties are cleaved from the signal-generating moieties.

2. A target-specific magnetic-resonance-imaging (“MRI”) contrast-enhancing agent comprising an extended poly(amino acid), wherein at least 90 percent of amino acid residues of the poly(amino acid) are conjugated to chelating moieties that form coordination complexes with paramagnetic ions, which are substantially shielded from surrounding water molecules by blocking moieties that are attached to the chelating moieties via linkers that are cleavable by a physiological target substance produced by a target; the target-specific MRI contrast-enhancing agent being rendered functionally more effective when the blocking moieties are cleaved from the chelating moieties.

3. The target-specific MRI contrast-enhancing agent according to claim 2; wherein the poly(amino acid) is selected from the group consisting of polylysine, polyhistidine, polyarginine, polyasparagine, polyglutamine, and copolymers of at least two types of amino acids that are selected from the group consisting of lysine, histidine, arginine, asparagine, glutamine, glutamic acid, and aspartic acid.

4. The MRI contrast-enhancing agent according to claim 3, wherein the chelating moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis{3-(4-carboxyl)-butanoic acid); 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

5. The MRI contrast-enhancing agent according to claim 3, wherein the poly(amino acid) comprises a number of amino acid residues in a range from about 100 to about 650.

6. The MRI contrast-enhancing agent according to claim 3, wherein the poly(amino acid) has a persistence length from about 100 to about 600 angstroms.

7. The MRI contrast-enhancing agent according to claim 3, wherein the chelating moieties are diethylene triamine pentaacetic acid.

8. The MRI contrast-enhancing agent according to claim 3, wherein the paramagnetic ions are selected from the group consisting of ions of transition metals, rare earth metals, and actinide elements.

9. The MRI contrast-enhancing agent according to claim 3, wherein the paramagnetic ions are selected from the group consisting of Gd³⁺, Dy³⁺, and a mixture thereof.

10. The MRI contrast-enhancing agent according to claim 3, wherein the physiological target substance comprises an enzyme that is overproduced by the target tissue, and the linkers comprise substrates for the enzyme, which substrates comprise peptides.

11. The MRI contrast-enhancing agent according to claim 10, wherein the enzyme is selected from the group consisting of proteases, carbohydrases, lipases, nucleases, tautomerases, mutases, and transferases.

12. The MRI contrast-enhancing agent according to claim 11, wherein the proteases are selected from the group consisting of matrix metalloproteases, caspases, and cathepsins; and wherein the target tissue comprises a tumor.

13. The MRI contrast-enhancing agent according to claim 12; wherein the proteases are selected from the group consisting of matrix metalloproteases; and wherein the linkers are peptides sequences selected from the group consisting of Pro-Leu-Gly-Val-Arg (SEQ ID NO. 1); Pro-Cha-Gly-Cys-His (SEQ ID NO. 2); Pro-Gln-Gly-Ile-Ala-Gly-Gln-D-Arg (SEQ ID NO. 3); Pro-Gln-Gly-Ile-Ala-Gly-Trp (SEQ ID NO. 4); Pro-Leu-Gly-Cys-His-Ala-D-Arg (SEQ ID No. 5); Pro-Leu-Gly-Met-Trp-Ser-Arg (SEQ ID. NO. 6); Pro-Leu-Gly-Leu-Trp-Ala-D-Arg (SEQ ID NO. 7); Pro-Leu-Ala-Leu-Trp-Ala-Arg (SEQ ID. NO. 8); Pro-Leu-Ala-Leu-Trp-Ala-Arg (SEQ ID NO. 9); Pro-Leu-Ala-Tyr-Trp-Ala-Arg (SEQ ID NO. 10); Pro-Tyr-Ala-Tyr-Trp-Met-Arg (SEQ ID NO. 11); Pro-Cha-Gly-Nva-His-Ala (SEQ ID NO. 12); Pro-Leu-Ala-Nva (SEQ ID NO. 13); Pro-Leu-Gly-Leu (SEQ ID NO. 14); Pro-Leu-Gly-Ala (SEQ ID NO. 15); Arg-Pro-Leu-Ala-Leu-Trp-Arg-Ser (SEQ ID NO. 16); Pro-Cha-Ala-Abu-Cys-His-Ala (SEQ ID NO. 17); Pro-Cha-Ala-Gly-Cys-His-Ala (SEQ ID NO. 18); Pro-Lys-Pro-Gln-Gln-Phe-Phe-Gly-Leu (SEQ ID NO. 19); Pro-Lys-Pro-Leu-Ala-Leu (SEQ ID NO. 20); Arg-Pro-Lys-Pro-Tyr-Ala-Nva-Trp-Met (SEQ ID NO. 21); Arg-Pro-Lys-Pro-Val-Glu-Nva-Trp-Arg (SEQ ID NO. 22); Arg-Pro-Lys-Pro-Val-Glu-Nva-Trp-Arg (SEQ ID NO. 23); and Arg-Pro-Lys-Pro-Leu-Ala-Nva-Trp (SEQ ID NO. 24); wherein Abu is L-α-aminobutyryl, Cha is L-cyclohexylalanine, and Nva is L-norvaline.

14. The MRI contrast-enhancing agent according to claim 12; wherein the proteases are selected from the group consisting of caspases; and wherein the linkers are peptides sequences selected from the group consisting of Phe-Glu-Ala-Asp SEQ ID NO. 25); Tyr-Val-His-Asp (SEQ ID NO. 26); Leu-Glu-Ser-Asp (SEQ ID NO. 27); Asp-Glu-Val-Asp (SEQ ID NO. 28); Asp-Gly-Pro-Asp (SEQ ID NO. 29); Asp-Glu-Leu-Asp (SEQ ID NO. 30); Asp-Glu-Glu-Asp (SEQ ID NO. 31); and Val-Glu-Ile-Asp (SEQ ID NO. 32).

15. The MRI contrast-enhancing agent according to claim 12;
wherein the enzyme is cathepsin D, and the linkers comprise a peptide sequence of Pro-Ile-Cys-Phe-Phe-Arg-Leu (SEQ ID NO. 33).

16. The MRI contrast-enhancing agent according to claim 3, further comprising a target-specific ligand that is capable of binding to at least a receptor on cells of the target tissue.

17. The MRI contrast-enhancing agent according to claim 16; wherein the target-specific ligand is selected from the group consisting of linear peptide sequence EMTOVNOG (SEQ ID NO. 34) and cyclic peptide EMTOVNOGQ (SEQ ID NO. 35), the cells are MCF-7 human breast cancer cells, and the receptor is α-fetoprotein (“AFP”) receptor, wherein E, M, T, V, N, G, Q, and O represent glutamic acid, methionine, threonine, valine, asparagines, glycine, glutamine, and 4-hydroxyproline, respectively.

18. The MRI contrast-enhancing agent according to claim 3, further comprising at least a therapeutic moiety that is attached to the MRI contrast-enhancing agent via a linker that is cleavable by the physiological target substance.

19. The MRI contrast-enhancing agent according to claim 2; wherein the poly(amino acid) is a copolymer of at least a first type of amino acid selected from the group consisting of lysine, histidine, arginine, asparagine, and glutamine; and at least a second type of amino acid selected from the group consisting of glutamic acid and aspartic acid.

20. The MRI contrast-enhancing agent according to claim 19, wherein the chelating moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

21. The MRI contrast-enhancing agent according to claim 19, wherein the poly(amino acid) comprises a number of amino acid residues in a range from about 100 to about 650.

22. The MRI contrast-enhancing agent according to claim 19, wherein the poly(amino acid) has a persistence length from about 100 to about 600 angstroms.

23. The MRI contrast-enhancing agent according to claim 19, wherein the chelating moieties are diethylene triamine pentaacetic acid.

24. The MRI contrast-enhancing agent according to claim 19, wherein the paramagnetic ions are selected from the group consisting of ions of transition metals, rare earth metals, and actinide elements.

25. The MRI contrast-enhancing agent according to claim 19, wherein the paramagnetic ions are selected from the group consisting of Gd³⁺, Dy³⁺, and a mixture thereof.

26. The MRI contrast-enhancing agent according to claim 19, wherein the physiological substance is an enzyme that is overproduced by the target tissue, and the linkers are substrates for the enzyme, which substrates comprise peptides.

27. The MRI contrast-enhancing agent according to claim 26, wherein the enzyme is selected from the group consisting of proteases, carbohydrases, lipases, nucleases, isomerases, epimerases, tautomerases, mutases, transferases, kinases, and phosphatases.

28. The MRI contrast-enhancing agent according to claim 27, wherein the proteases are selected from the group consisting of matrix metalloproteases, caspases, and cathepsins; and wherein the target tissue comprises a tumor.

29. The MRI contrast-enhancing agent according to claim 28; wherein the proteases are selected from the group consisting of matrix metalloproteases; and wherein the linkers are peptides sequences selected from the group consisting of Pro-Leu-Gly-Val-Arg (SEQ ID NO. 1); Pro-Cha-Gly-Cys-His (SEQ ID NO. 2); Pro-Gln-Gly-Ile-Ala-Gly-Gln-D-Arg (SEQ ID NO. 3); Pro-Gln-Gly-Ile-Ala-Gly-Trp (SEQ ID NO. 4); Pro-Leu-Gly-Cys-His-Ala-D-Arg (SEQ ID No. 5); Pro-Leu-Gly-Met-Trp-Ser-Arg (SEQ ID. NO. 6); Pro-Leu-Gly-Leu-Trp-Ala-D-Arg (SEQ ID NO. 7); Pro-Leu-Ala-Leu-Trp-Ala-Arg (SEQ ID. NO. 8); Pro-Leu-Ala-Leu-Trp-Ala-Arg (SEQ ID NO. 9); Pro-Leu-Ala-Tyr-Trp-Ala-Arg (SEQ ID NO. 10); Pro-Tyr-Ala-Tyr-Trp-Met-Arg (SEQ ID NO. 11); Pro-Cha-Gly-Nva-His-Ala (SEQ ID NO. 12; Pro-Leu-Ala-Nva (SEQ ID NO. 13); Pro-Leu-Gly-Leu (SEQ ID-NO. 14); Pro-Leu-Gly-Ala (SEQ ID NO. 15); Arg-Pro-Leu-Ala-Leu-Trp-Arg-Ser (SEQ ID NO. 16); Pro-Cha-Ala-Abu-Cys-His-Ala (SEQ ID NO. 17); Pro-Cha-Ala-Gly-Cys-His-Ala (SEQ ID NO. 18); Pro-Lys-Pro-Gln-Gln-Phe-Phe-Gly-Leu (SEQ ID NO. 19); Pro-Lys-Pro-Leu-Ala-Leu (SEQ ID NO. 20); Arg-Pro-Lys-Pro-Tyr-Ala-Nva-Trp-Met (SEQ ID NO. 21); Arg-Pro-Lys-Pro-Val-Glu-Nva-Trp-Arg (SEQ ID NO. 22); Arg-Pro-Lys-Pro-Val-Glu-Nva-Trp-Arg (SEQ ID NO. 23); and Arg-Pro-Lys-Pro-Leu-Ala-Nva-Trp (SEQ ID NO. 24); wherein Abu is L-α-aminobutyryl, Cha is L-cyclohexylalanine, and Nva is L-norvaline.

30. The MRI contrast-enhancing agent according to claim 28; wherein the proteases are selected from the group consisting of caspases; and wherein the linkers are peptides sequences selected from the group consisting of Phe-Glu-Ala-Asp SEQ ID NO. 25); Tyr-Val-His-Asp (SEQ ID NO. 26); Leu-Glu-Ser-Asp (SEQ ID NO. 27); Asp-Glu-Val-Asp (SEQ ID NO. 28); Asp-Gly-Pro-Asp (SEQ ID NO. 29); Asp-Glu-Leu-Asp (SEQ ID NO. 30); (SEQ ID NO. 31); and Val-Glu-Ile-Asp (SEQ ID NO. 32).

31. The MRI contrast-enhancing agent according to claim 28; wherein the enzyme is cathepsin D, and the linkers comprise a peptide sequence of Pro-Ile-Cys-Phe-Phe-Arg-Leu (SEQ ID NO. 33).

32. The MRI contrast-enhancing agent according to claim 19, further comprising a target-specific ligand that is capable of binding to at least a receptor on cells of the target tissue.

33. The MRI contrast-enhancing agent according to claim 32;
wherein the target-specific ligand is selected from the group consisting of linear peptide sequence EMTOVNOG (SEQ ID NO. 34) and cyclic peptide EMTOVNOGQ (SEQ ID NO. 35), the cells are MCF-7 human breast cancer cells, and the receptor is α-fetoprotein (“AFP”) receptor, wherein E, M, T, V, N, G, Q, and O represent glutamic acid, methionine, threonine, valine, asparagines, glycine, glutamine, and 4-hydroxyproline, respectively.

34. The MRI contrast-enhancing agent according to claim 19, further comprising at least a therapeutic moiety that is attached to the MRI contrast-enhancing agent via a linker that is cleavable by the physiological target substance.

35. The target-specific image-enhancing agent according to claim 1; wherein the signal-generating moieties comprise a fluorescent dye, the physiological target substance comprises an enzyme, and the signal-controlling moieties comprise substrates for the enzyme, which substrates comprise peptides.

36. The target-specific image-enhancing agent according to claim 35; wherein the fluorescent dye is attached to polycarboxylic acid moieties via the signal-controlling moieties, and each of the polycarboxylic acid moieties is conjugated to an amino acid residue of the poly(amino acid).

37. The target-specific image-enhancing agent according to claim 36; wherein the poly(amino acid) is selected from the group consisting of polylysine, polyhistidine, polyarginine, polyasparagine, polyglutamine, and copolymers of at least two types of amino acids that are selected from the group consisting of lysine, histidine, arginine, asparagine, glutamine, glutamic acid, and aspartic acid.

38. The target-specific image-enhancing agent according to claim 37, wherein the polycarboxylic acid moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

39. The target-specific image-enhancing agent according to claim 37, wherein the poly(amino acid) comprises a number of amino acid residues in a range from about 100 to about 650.

40. The target-specific image-enhancing agent according to claim 37, wherein the poly(amino acid) has a persistence length from about 100 to about 600 angstroms.

41. The target-specific image-enhancing agent according to claim 37, wherein the chelating moieties are diethylene triamine pentaacetic acid.

42. The target-specific image-enhancing agent according to claim 37, wherein the enzyme is selected from the group consisting of proteases, carbohydrases, lipases, nucleases, tautomerases, mutases, and transferases.

43. The target-specific image-enhancing agent according to claim 42, wherein the proteases are selected from the group consisting of matrix metalloproteases, caspases, and cathepsins; and wherein the target comprises a tumor.

44. The target-specific image-enhancing agent according to claim 43; wherein the proteases are selected from the group consisting of matrix metalloproteases; and wherein the linkers are peptides sequences selected from the group consisting of Pro-Leu-Gly-Val-Arg (SEQ ID NO. 1); Pro-Cha-Gly-Cys-His (SEQ ID NO. 2); Pro-Gln-Gly-Ile-Ala-Gly-Gln-D-Arg (SEQ ID NO. 3); Pro-Gln-Gly-Ile-Ala-Gly-Trp (SEQ ID NO. 4); Pro-Leu-Gly-Cys-His-Ala-D-Arg (SEQ ID No. 5); Pro-Leu-Gly-Met-Trp-Ser-Arg (SEQ ID. NO. 6); Pro-Leu-Gly-Leu-Trp-Ala-D-Arg (SEQ ID NO. 7); Pro-Leu-Ala-Leu-Trp-Ala-Arg (SEQ ID. NO. 8); Pro-Leu-Ala-Leu-Trp-Ala-Arg (SEQ ID NO. 9); Pro-Leu-Ala-Tyr-Trp-Ala-Arg (SEQ ID NO. 10); Pro-Tyr-Ala-Tyr-Trp-Met-Arg (SEQ ID NO. 11); Pro-Cha-Gly-Nva-His-Ala (SEQ ID NO. 12); Pro-Leu-Ala-Nva (SEQ ID NO. 13); Pro-Leu-Gly-Leu (SEQ ID NO. 14); Pro-Leu-Gly-Ala (SEQ ID NO. 15); Arg-Pro-Leu-Ala-Leu-Trp-Arg-Ser (SEQ ID NO. 16); Pro-Cha-Ala-Abu-Cys-His-Ala (SEQ ID NO. 17); Pro-Cha-Ala-Gly-Cys-His-Ala (SEQ ID NO. 18); Pro-Lys-Pro-Gln-Gln-Phe-Phe-Gly-Leu (SEQ ID NO. 19); Pro-Lys-Pro-Leu-Ala-Leu (SEQ ID NO. 20); Arg-Pro-Lys-Pro-Tyr-Ala-Nva-Trp-Met (SEQ ID NO. 21); Arg-Pro-Lys-Pro-Val-Glu-Nva-Trp-Arg (SEQ ID NO. 22); Arg-Pro-Lys-Pro-Val-Glu-Nva-Trp-Arg (SEQ ID NO. 23); and Arg-Pro-Lys-Pro-Leu-Ala-Nva-Trp (SEQ ID NO. 24); wherein Abu is L-α-aminobutyryl, Cha is L-cyclohexylalanine, and Nva is L-norvaline.

45. The target-specific image-enhancing agent according to claim 43; wherein the proteases are selected from the group consisting of caspases; and wherein the linkers are peptides sequences selected from the group consisting of Phe-Glu-Ala-Asp SEQ ID NO. 25); Tyr-Val-His-Asp (SEQ ID NO. 26); Leu-Glu-Ser-Asp (SEQ ID NO. 27); Asp-Glu-Val-Asp (SEQ ID NO. 28); Asp-Gly-Pro-Asp (SEQ ID NO. 29); Asp-Glu-Leu-Asp (SEQ ID NO. 30); Asp-Glu-Glu-Asp (SEQ ID NO. 31); and Val-Glu-Ile-Asp (SEQ ID NO. 32).

46. The target-specific image-enhancing agent according to claim 43; wherein the enzyme is cathepsin D, and the linkers comprise a peptide sequence of Pro-Ile-Cys-Phe-Phe-Arg-Leu (SEQ ID NO. 33).

47. The target-specific image-enhancing agent according to claim 37, further comprising a target-specific ligand that is capable of binding to at least a receptor on cells of the target tissue.

48. The target-specific image-enhancing agent according to claim 47; wherein the target-specific ligand is selected from the group consisting of linear peptide sequence EMTOVNOG (SEQ ID NO. 34) and cyclic peptide EMTOVNOGQ (SEQ ID NO. 35), the cells are MCF-7 human breast cancer cells, and the receptor is α-fetoprotein (“AFP”) receptor, wherein E, M, T, V, N, G, Q, and O represent glutamic acid, methionine, threonine, valine, asparagines, glycine, glutamine, and 4-hydroxyproline, respectively.

49. The target-specific image-enhancing agent of claim 35, further comprising at least a therapeutic moiety that is attached to the target-specific image-enhancing agent via at least a linker that is cleavable by the physiological target substance.

50. A method for detecting a disease condition, the method comprising:
administering into a subject at least an imaging agent that is activatable by an expression of the disease; the imaging agent comprising: (1) an extended poly(amino acid), wherein at least 90 percent of amino acid residues of the poly(amino acid) are conjugated to signal-generating moieties; and (2) a plurality of cleavable signal-controlling moieties attached to the signal-generating moieties via bonds that are cleavable substantially at a target by a physiological target substance produced by the target;
obtaining images, before and after the step of administering the imaging agent, of a portion of the body of the subject, which portion is suspected of carrying the disease; and
comparing the images obtained before the step of administering to the images obtained after the step of administering to identify an area showing an increase in a signal generated by an activation of the imaging agent, which indicates a disease condition.

51. The method according to claim 50, wherein the poly(amino acid) is selected from the group consisting of homopolymers and copolymers of amino acid residues.

52. The method according to claim 50, wherein the poly(amino acid) is poly-L-lysine.

53. The method according to claim 50, wherein the poly(amino acid) is poly(glutamic acid).

54. The method according to claim 50, wherein the poly(amino acid) comprises a number of amino acid residues in a range from about 100 to about 650.

55. The method according to claim 50, wherein the poly(amino acid) has a persistence length in a range from about 100 to about 600 angstroms.

56. The method according to claim 50, wherein the poly(amino acid) is selected from the group consisting of polyhistidine, polyarginine, polyasparagine, polyglutamine, and copolymers of at least two types of amino acids selected from the group consisting of lysine, histidine, arginine, asparagine, glutamine, glutamic acid, and aspartic acid.

57. The method according to claim 50, wherein the poly(amino acid) is a copolymer of glutamic acid and aspartic acid.

58. The method according to claim 50, wherein the poly(amino acid) is a copolymer of at least a first type of amino acid selected from the group consisting of lysine, histidine, arginine, asparagine, and glutamine; and at least a second type of amino acid selected from the group consisting of glutamic acid and aspartic acid.

59. The method according to claim 50; wherein the imaging agent is at least an MRI contrast-enhancing agent; the signal-generating moieties comprise chelating moieties that are capable of forming coordination complexes with paramagnetic ions; and the chelating moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis(3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

60. The method according to claim 59, wherein the chelating moieties are diethylene triamine pentaacetic acid.

61. The method according to claim 59, wherein the paramagnetic ions are selected from the group consisting of ions of transition metal elements, rare-earth metal elements, and actinide elements.

62. The method according to claim 59, wherein the paramagnetic ions are selected from the group consisting of Gd³⁺, Dy³⁺, and a mixture thereof.

63. The method according to claim 59, wherein said at least an MRI contrast-enhancing agent is administered into the subject at a dose in a range from about 0.01 to about 0.5 mole Gd/kg of body weight of the subject.

64. The method according to claim 59, wherein the images obtained after the step of administering are obtained within 48 hours after said administering.

65. A method for assessing an effectiveness of a prescribed regimen for treating a disease that is characterized by an overproduction of a disease-specific substance, the method comprising:
(a) obtaining at least a base-line image of and acquiring a base-line signal from a portion of a subject, which portion is suspected to carry the disease;
(b) administering a first time into a subject a predetermined dose of at least an image-enhancing agent that comprises an extended poly(amino acid), at least 90 percent of amino acid residues of the poly(amino acid) being conjugated to signal-generating moieties;
(c) obtaining pre-treatment images of and acquiring pre-treatment signals coming from the portion of the subject, after administering the predetermined dose of the image-enhancing agent into the subject;
(d) treating a condition of the disease in the subject with the prescribed regimen;
(e) administering a second time into the subject the predetermined dose of said at least an image-enhancing agent;
(f) obtaining post-treatment images of and acquiring post-treatment signals coming from the same portion of the subject as in step (c); and
(g) comparing post-treatment images and post-treatment signals to pre-treatment images and pre-treatment signals to assess the effectiveness of the prescribed regimen; a decrease in image contrast or signals during the course of the prescribed regimen indicating that the treatment has provided benefit.

66. A method for detecting and treating a disease condition, the method comprising:
(a) administering into a subject at least an imaging-and-therapeutic agent that is activatable by an expression of the disease; the imaging-and-therapeutic agent comprising: (1) an extended poly(amino acid), wherein at least 90 percent of amino acid residues of the poly(amino acid) are conjugated to signal-generating moieties; (2) a plurality of cleavable signal-controlling moieties attached to the signal-generating moieties via bonds that are cleavable substantially at a target by a physiological target substance produced by the target; and (3) at least a therapeutic moiety attached to the imaging-and-therapeutic agent via a bond that is cleavable substantially at the target by the physiological target substance;
(b) obtaining images, before and after the step of administering the imaging-and-therapeutic agent, of a portion of the body of the subject, which portion is suspected of carrying the disease; and
(c) comparing the images obtained before the step of administering to the images obtained after the step of administering to identify an area showing a change in a signal generated by an activation of the imaging-and-therapeutic agent, which indicates a disease condition.


SEQUENCE LISTING (35 sequences)

SEQ ID NO: 1 - protein, 5 aa, artificial sequence
  a peptide substrate for a metalloprotease
PLGVR

SEQ ID NO: 2 - protein, 5 aa, artificial sequence
  a substrate for a metalloprotease
PXGCH

SEQ ID NO: 3 - protein, 7 aa, artificial sequence
  a substrate for a metalloprotease
PQGIAGX

SEQ ID NO: 4 - protein, 7 aa, artificial sequence
  a substrate for a metalloprotease
PQGIAGW

SEQ ID NO: 5 - protein, 7 aa, artificial sequence
  a substrate for a metalloprotease
PLGCHAX

SEQ ID NO: 6 - protein, 7 aa, artificial sequence
  a substrate for a metalloprotease
PLGMWSR

SEQ ID NO: 7 - protein, 6 aa, artificial sequence
  a substrate for a metalloprotease
LGLWAX

SEQ ID NO: 8 - protein, 7 aa, artificial sequence
  a substrate for a metalloprotease
PLALWAA

SEQ ID NO: 9 - protein, 7 aa, artificial sequence
  a substrate for a metalloprotease
PLALWAR

SEQ ID NO: 10 - protein, 7 aa, artificial sequence
  a substrate for a mettaloprotease
PLAYWAR

SEQ ID NO: 11 - protein, 7 aa, artificial sequence
  a substrate for a metalloprotease
PYAYWMR

SEQ ID NO: 12 - protein, 6 aa, artificial sequence
  a substrate for a metalloprotease
PXGXHA

SEQ ID NO: 13 - protein, 4 aa, artificial sequence
  a substrate for a metalloprotease
PLAX

SEQ ID NO: 14 - protein, 4 aa, artificial sequence
  a substrate for a metalloprotease
PLGL

SEQ ID NO: 15 - protein, 4 aa, artificial sequence
  a substrate for a metalloprotease
PLGA

SEQ ID NO: 16 - protein, 8 aa, artificial sequence
  a substrate for a metalloprotease
RPLALWRS

SEQ ID NO: 17 - protein, 7 aa, artificial sequence
  a substrate for a metalloprotease
PXAXCHA

SEQ ID NO: 18 - protein, 7 aa, artificial sequence
  a substrate for a metalloprotease
PXAGCHA

SEQ ID NO: 19 - protein, 9 aa, artificial sequence
  a substrate for a metalloprotease
PKPQQFFGL

SEQ ID NO: 20 - protein, 6 aa, artificial sequence
  a substrate for a metalloprotease
PKPLAL

SEQ ID NO: 21 - protein, 9 aa, artificial sequence
  a substrate for a metalloprotease
RPKPYAXWM

SEQ ID NO: 22 - protein, 9 aa, artificial sequence
  a substrate for a metalloprotease
RPKPVEXWR

SEQ ID NO: 23 - protein, 9 aa, artificial sequence
  a substrate for a metalloprotease
RPKPVEXWR

SEQ ID NO: 24 - protein, 8 aa, artificial sequence
  a substrate for a metalloprotease
RPKPLAXW

SEQ ID NO: 25 - protein, 4 aa, artificial sequence
  a substrate for caspase-1
FEAD

SEQ ID NO: 26 - protein, 4 aa, artificial sequence
  a substrate for caspase-1
YVHD

SEQ ID NO: 27 - protein, 4 aa, artificial sequence
  a substrate for caspase-1
LESD

SEQ ID NO: 28 - protein, 4 aa, artificial sequence
  a substrate for caspase-3
DEVD

SEQ ID NO: 29 - protein, 4 aa, artificial sequence
  a substrate for caspase-3
DGPD

SEQ ID NO: 30 - protein, 4 aa, artificial sequence
  a substrate for caspase-3
DELD

SEQ ID NO: 31 - protein, 4 aa, artificial sequence
  a substrate for caspase-3
DEED

SEQ ID NO: 32 - protein, 4 aa, artificial sequence
  a substrate for caspase-6
VEID

SEQ ID NO: 33 - protein, 7 aa, artificial sequence
  a substrate for cathepsin D
PICFFRL

SEQ ID NO: 34 - protein, 8 aa, artificial sequence
  a linear peptide binding to alpha-fetoprotein receptor on MCF-7 human breast cancer cells
EMTXVNXG

SEQ ID NO: 35 - protein, 9 aa, artificial sequence
  a cyclic peptide binding to alpha-fetoprotein receptor on MCF-7 human breast cancer cells
EMTXVNXGQ